(19) United States
(12) Patent Application Publication
(10) Pub. No.: US 2021/0069240 A1
(43) Pub. Date: Mar. 11, 2021

(54) MATERIALS AND METHODS FOR BLOOD PLASMA PREPARATIONS

(71) Applicant: Cellphire, Inc., Rockville, MD (US)

(72) Inventors: Rafael Jorda, Bethesda, MD (US); Anne S. Hale, Placitas, NM (US); Stephen Edward Amos, Buckeystown, MD (US); Benjamin J. Kuhn, Norfolk, VA (US)

(21) Appl. No.: 17/013,091

(22) Filed: Sep. 4, 2020

Related U.S. Application Data
(60) Provisional application No. 62/896,249, filed on Sep. 5, 2019.

(51) Int. Cl. A61K 35/16; A61J 1/10; A61K 9/16

(52) U.S. Cl. CPC A61K 35/16; A61K 9/1682; A61J 1/10

(57) ABSTRACT

Provided herein in some embodiments is a method of preparing a plasma product, including providing plasma, acidifying the plasma to form acidified plasma, drying the acidified plasma to form dried plasma, and milling the dried plasma to form a plasma product. Also provided are plasma products produced by the methods described herein.

### CROSS-REFERENCE TO RELATED APPLICATIONS

This application claims priority to U.S. Application Ser. No. 62/896,249, filed on Sep. 5, 2019, which is incorporated by reference herein in its entirety.

### STATEMENT OF GOVERNMENT INTEREST

This invention was made with government support under Contract Nos. H92222-17-P-0008 and W911NF-18-C-0087 awarded by the United States Special Operations Command. The government has certain rights in the invention.

### FIELD OF THE INVENTION

The present disclosure in some embodiments relates to blood plasma products, such as dried plasma products.

### BACKGROUND

Blood is a complex mixture of numerous components. In general, blood can be described as comprising four main parts: red blood cells, white blood cells, platelets, and plasma. The first three are cellular or cell-like components, whereas the fourth (plasma) is a liquid component comprising a wide and variable mixture of salts, proteins, and other factors necessary for numerous bodily functions. The components of blood can be separated from each other by various methods. In general, differential centrifugation is most commonly used currently to separate the different components of blood based on size and, in some applications, density.

Military Working Dogs have proven to be a vital component of significant warfighter missions, supporting warfighter security and mission implementation. A lack of appropriate canine transfusion products risks the lives of these dogs, as well as the success of the mission, when a dog suffers severe trauma in the field. Plasma is increasingly recognized as a life-saving product for severe trauma offering advantages over transfusion of blood alone. However, the use of plasma in battlefield situations is limited by logistical constraints.

### SUMMARY OF THE INVENTION

Provided herein in some embodiments is a method of preparing a plasma product, the method including providing canine plasma, acidifying the canine plasma to form acidified plasma, drying the acidified plasma to form dried plasma, and milling the dried plasma to form a plasma product.

Implementations can include one or more of the following features. The canine plasma can be pooled plasma. Acidifying the canine plasma can include contacting the canine plasma with about 0.5% to about 2.5% (v/v) of a 1N acid. Acidifying the canine plasma can include contacting the canine plasma with about 1.25% to about 1.7% (v/v) of a 1N acid. The acid can be a mineral acid. The acid can be hydrochloric acid. Drying the acidified plasma can include lyophilizing the acidified plasma. Lyophilizing can include using the lyophilization protocol in Table 1. Lyophilizing can include freezing the acidified plasma to below −5° C. to form frozen plasma, reducing the pressure to less than 100 mTorr, and drying the frozen plasma to form dried plasma. Drying the frozen plasma can occur at atmospheric pressure. Drying the frozen plasma can include a starting temperature of below 0° C. The starting temperature can be about −5° C. Drying the frozen plasma can include an ending temperature above 0° C. The ending temperature can be about 25° C. The freezing step can have a duration of at least about 1 hour. The drying step can have a duration of at least 24 hours. The drying step can have a duration of at least 48 hours. The drying step can have a duration of at least 72 hours. The drying step can include a primary drying step and a secondary drying step. Drying the acidified plasma can include spray drying the acidified plasma. The method can further include rehydrating the plasma product to form a rehydrated plasma product. Rehydrating can include rehydrating with water. Rehydrating can include rehydrating with a buffer. Rehydrating the plasma product can include contacting the plasma product with a mass of water or buffer about 10-fold to about 20-fold of the mass of the plasma product. Rehydrating the plasma product can include contacting the plasma product with a mass of water or buffer about 12-fold to about 18-fold of the mass of the plasma product. Rehydrating the plasma product can include contacting the plasma product with a mass of water or buffer about 14-fold to about 16-fold of the mass of the plasma product. The rehydrated plasma product can have a fibrinogen concentration of at least about 150 mg/dL. The rehydrated plasma product can have a fibrinogen concentration of at least about 200 mg/dL. The rehydrated plasma product can have a fibrinogen concentration of at least about 250 mg/dL. The rehydrated plasma product can have a von Willebrand Factor antigen percentage of at least about 50%. The rehydrated plasma product can have a von Willebrand Factor antigen percentage of at least about 70%. The rehydrated plasma product can have a von Willebrand Factor antigen percentage of at least about 100%. The rehydrated plasma product can have a Factor VII coagulant activity of at least about 30%. The rehydrated plasma product can have a Factor VII coagulant activity of at least about 50%. The rehydrated plasma product can have a Factor VII coagulant activity of at least about 100%. The rehydrated plasma product can have a Factor VIII coagulant activity of at least about 50%. The rehydrated plasma product can have a Factor VIII coagulant activity of at least about 70%. The rehydrated plasma product can have a Factor VIII coagulant activity of at least about 100%. The rehydrated plasma product can have an albumin concentration of at least about 1.5 mg/dL. The rehydrated plasma product can have an albumin concentration of at least about 2.2 mg/dL. The rehydrated plasma product can have an albumin concentration of at least about 2.5 mg/dL. The rehydrated plasma product can have a pH of at least about 5.5. The rehydrated plasma product can have a pH between about 5.5 and about 7.8. The rehydrated plasma product can have a pH of at least about 6.0. The rehydrated plasma product can have a pH of between about 6.0 and about 7.8. The rehydrated plasma product can have a pH of at least about 6.5. The rehydrated plasma product can have a pH of between about 6.5 and about 7.8. The rehydrated plasma product can have a pH of about 7.0 to about 7.8. The rehydrated plasma product can have a pH of about 7.2 to about 7.6. Rehydrating can include rehydrating over a period of between 1 and 3 minutes. Rehydrating can include rehydrating over a period of less than 2 minutes. Rehydrating can include rehydrating over a period of less than 1 minute. Rehydrating can include rehydrating over a period of 1-2 minutes. The plasma product can be stable for at least two years at a temperature of about 15° F. to about 140° F. The plasma product can be stable for at least two years at a temperature of about −9.5° C. to about 60° C.

In some embodiments, also provided herein is a plasma product produced by any of the methods described herein.

In some embodiments, also provided herein is a rehydrated plasma product produced by any of the methods described herein.

In some embodiments, provided herein is a method of treating a canine subject, the method including rehydrating a plasma product produced by any of the methods described herein to form a rehydrated plasma product, and administering the rehydrated plasma product to the subject. In some embodiments, provided herein is a method of treating a canine subject, the method including administering a rehydrated plasma product produced by any of the methods described herein to the subject. In some embodiments, subject can have undergone trauma. In some embodiments, the subject can have a coagulopathy.

### DESCRIPTION OF THE DRAWINGS

FIG. 1 is an exemplary schematic of a process for the preparation of plasma products according to some embodiments of the current disclosure.

FIG. 2A is a plot of the pH of post-rehydration plasma prepared by using about 0.5% to about 2.5% (v/v) hydrochloric acid in pre-lyophilization plasma.

FIG. 2B is a plot of the pH of post-rehydration plasma prepared by using about 1.25% to about 1.7% (v/v) hydrochloric acid in pre-lyophilization plasma.

FIG. 3 is an exemplary product produced by the methods described herein, showing (1) syringe access, (2) an administration line with a small volume filter, and (3) a rehydration port.

FIG. 4 shows an exemplary jar mill.

### DESCRIPTION OF ATTACHMENTS

Appendix 1 is a document entitled, “PRESCRIBING INFORMATION FOR StablePlas RX™” undated and not made public as of the filing of this provisional patent application (Sep. 5, 2019). It does not necessarily reflect final prescribing information for any product. This document is incorporated by reference into the present patent application document.

### DETAILED DESCRIPTION

It is to be understood that the terminology used herein is for the purpose of describing particular embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9,1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this document by the term “about”.

Unless defined otherwise, all technical and scientific terms used herein have the same meaning as commonly understood by one of ordinary skill in the art to which the term belongs. Although any methods and materials similar or equivalent to those described herein can be used in the practice or testing of the present disclosure, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “a platelet” includes a plurality of such platelets. Furthermore, the use of terms that can be described using equivalent terms include the use of those equivalent terms. Thus, for example, the use of the term “subject” is to be understood to include the terms “patient”, “individual” and other terms used in the art to indicate one who is subject to a treatment.

As used herein, “plasma” refers to the plasma component of blood, significantly separated from other components of whole blood, for example, by centrifugation or plasmapheresis.

As used herein, an “exogenous” polymer or “exogenous” saccharide in plasma refers to a polymer or saccharide, respectively, that was not in the plasma or whole blood from which the plasma was derived when taken from a donor.

Maintaining stability of plasma components often requires refrigeration or freezing, and the sheer bulk of the product hinders transport and distribution. However, plasma products, such as plasma products provided herein, can provide a transfusion product with reduced volume, elimination of the need for cold storage, and the potential to concentrate anti-inflammatory molecules which may provide an added advantage. An additional advantage which may be present in the plasma products described herein is that the plasma products can have a reconstitution (also called rehydration herein) protocol off the shelf with rehydration in less than 2 minutes in a fit-for-function form ready for immediate transfusion. Another potential advantage of the plasma products of the disclosure is the retention, after rehydration, functional levels of one or more key hemostatic proteins relative to fresh frozen plasma. Another potential advantage of the plasma products of the disclosure is an appropriate post-hydration pH (e.g., for transfusion).

Accordingly, provided herein are methods of preparing a plasma product. The method can include providing plasma, acidifying the plasma to form acidified plasma, drying the acidified plasma to form dried plasma, and milling the dried plasma to form a plasma product. In some embodiments, the method can consist of providing plasma, acidifying the plasma to form acidified plasma, drying the acidified plasma to form dried plasma, and milling the dried plasma to form a plasma product. In some embodiments, the method can consist essentially of providing plasma, acidifying the plasma to form acidified plasma, drying the acidified plasma to form dried plasma, and milling the dried plasma to form a plasma product, such that additional steps may be performed that do not result in a significant difference in the plasma product from a method consisting of the aforementioned steps; for example, dilution, incubation, and heating or cooling steps may be performed such that they do not result in a significant difference in the plasma product from a method consisting of the aforementioned steps. In some embodiments, a significant difference can be determined using one or more of the stability measures described herein.

Any appropriate source(s) of plasma can be used. Typically, plasma from a single species is used. In some embodiments, canine plasma can be used. In some embodiments, feline plasma can be used. In some embodiments, equine plasma can be used. In some embodiments, bovine plasma can be used. In some embodiments, porcine plasma can be used. In some embodiments, simian plasma can be used. In some embodiments, human plasma can be used. In some embodiments, the plasma is pooled from a plurality of donor subjects. Such plasma pooled from a plurality of donor subjects may be also referred herein to as pooled plasma. In some embodiments, the donor subjects are more than 5, such as more than 10, such as more than 20, such as more than 50, such as up to about 100 donor subjects. In some embodiments, the donor subjects are from about 5 to about 100, such as from about 10 to about 50, such as from about 20 to about 40, such as from about 25 to about 35 donor subjects.

Acidifying plasma can be carried out using any appropriate method. In some embodiments, acidifying plasma can be carried out by adding an acid to the plasma (e.g., pooled plasma). In some embodiments, the acid can be a mineral acid. In some embodiments, the acid can be hydrochloric acid (HCl). In some embodiments, the acid can be HCl, nitric acid (HNO₃), hydroiodic acid (HI), chloric acid (HClO₃), or perchloric acid (HClO₄) In some embodiments, acidifying the plasma can include contacting the plasma with about 0.5% to about 2.5% (v/v) (e.g., about 1.0% to about 2.0% (v/v), about 1.25% to about 1.75% (v/v), or about 1.25% to about 1.7% (v/v)) of a 1N acid. Without being bound by any particular theory, it is believed that the amount of acid used for acidifying the plasma is correlated with the post-rehydration pH of a rehydrated plasma product.

In some embodiments, methods of preparing a plasma product as described herein can exclude certain steps or components. For example, in some embodiments, a method of preparing a plasma product does not include addition of platelets or platelet derivatives to plasma, acidified plasma, dried plasma, or plasma product. In some embodiments, a method of preparing a plasma product does not include addition of red blood cells (and/or red blood cell substitutes) to plasma, acidified plasma, dried plasma, or plasma product. In some embodiments, a method of preparing a plasma product does not include addition of components other than acid to plasma. In some embodiments, a method of preparing a plasma product does not include addition of components to acidified plasma, dried plasma, or plasma product. In some embodiments, the plasma does not include an exogenous polymer. In some embodiments, the plasma does not include an exogenous saccharide. In some embodiments, the acidified plasma does not include an exogenous polymer. In some embodiments, the acidified plasma does not include an exogenous saccharide. In some embodiments, the dried plasma does not include an exogenous polymer. In some embodiments, the dried plasma does not include an exogenous saccharide. In some embodiments, the plasma product does not include an exogenous polymer. In some embodiments, the plasma product does not include an exogenous saccharide.

Any known technique for drying plasma can be used in accordance with the present disclosure. In some cases, the technique used can achieve a final residual moisture content of less than 5%. Preferably, the technique achieves a final residual moisture content of less than 2%, such as 1%, 0.5%, or 0.1%. Non-limiting examples of suitable techniques are freeze-drying (lyophilization) and spray-drying. One exemplary lyophilization method is presented in Table A. A second exemplary lyophilization method is presented in Table 1. In some embodiments, lyophilizing can include freezing the acidified plasma to below about −5° C. to form frozen plasma, reducing the pressure to less than about 100 mTorr, and drying the frozen plasma to form dried plasma. In some embodiments, freezing the acidified plasma can occur at a temperature at or below −10° C. (e.g., at or below −20° C., −30° C., −40° C., −50° C., or −60° C.). In some embodiments, drying the frozen plasma can occur at atmospheric pressure. In some embodiments, drying the frozen plasma comprises a starting temperature of below 0° C. (e.g., at or below a temperature of −5° C., −10° C., or −20° C.). Such a temperature can be achieved, for example, by ramping from the temperature used for freezing the acidified plasma to the starting temperature for drying the frozen plasma. In some embodiments, drying the frozen plasma comprises an ending temperature above about 0° C. (e.g., at or above a temperature of about 5° C., 10° C., 15° C., 20° C., 25° C., or 30° C.). In some embodiments, the freezing step can have a duration of at least about 1 hour (e.g., at least about 2 hours). In some embodiments, the drying step can have a duration of at least about 24 hours (e.g., at least about 36, 48, 60, or 72 hours). In some embodiments, the drying step can include a primary drying step (e.g., including a primary starting temperature of below 0° C. (e.g., about −5° C.) and a primary ending temperature of above 0° C. (e.g., about 5° C.)) and a secondary drying step (e.g., including a secondary starting temperature that is approximately the same as the primary ending temperature (e.g., about 5° C.) and a secondary ending temperature above the primary ending temperature (e.g. about 25° C.)).

Additional exemplary lyophilization methods can be found in U.S. Pat. Nos. 7,811,558, 8,486,617, and 8,097,403.

An exemplary spray-drying method includes: combining nitrogen, as a drying gas, with acidified plasma as described herein to form a mixture, then introducing the mixture into GEA Mobile Minor spray dryer from GEA Processing Engineering, Inc. (Columbia Md., USA), which has a Two-Fluid Nozzle configuration, spray drying the mixture at an inlet temperature in the range of 150° C. to 190° C., an outlet temperature in the range of 65° C. to 100° C., an atomic rate in the range of 0.5 to 2.0 bars, an atomic rate in the range of 5 to 13 kg/hr, a nitrogen use in the range of 60 to 100 kg/hr, and a run time of 10 to 35 minutes. The final step in spray drying is preferentially collecting the dried mixture. The dried composition in some embodiments is stable for at least six months at temperatures that range from −20° C. or lower to 90° C. or higher.

TABLE A
Exemplary Lyophilization Protocol

| | Step | Temp. Set | | Type | Durati | | Pressure Set |
|---|---|---|---|---|---|---|---|
| Freezing Step | F1 | −50° | C. | Ramp | Var | | N/A |
| | F2 | −50° | C. | Hold | 3 | Hrs | N/A |
| Vacuum Pulldown | F3 | −50° | | Hold | Var | | N/A |
| Primary Dry | P1 | −40° | | Hold | 1.5 | Hrs | 0 mT |
| | P2 | −35° | | Ramp | 2 | Hrs | 0 mT |
| | P3 | −25° | | Ramp | 2 | Hrs | 0 mT |
| | P4 | −17° | C. | Ramp | 2 | Hrs | 0 mT |
| | P5 | 0° | C. | Ramp | 1.5 | Hrs | 0 mT |
| | P6 | 27° | C. | Ramp | 1.5 | Hrs | 0 mT |
| | P7 | 27° | C. | Hold | 16 | Hrs | 0 mT |
| Secondary Dry | S1 | 27° | C. | Hold | >8 | Hrs | 0 mT |

In various embodiments, the lyophilization bag is a gas-permeable bag configured to allow gases to pass through at least a portion or all portions of the bag during the processing. The gas-permeable bag can allow for the exchange of gas within the interior of the bag with atmospheric gas present in the surrounding environment. The gas-permeable bag can be permeable to gases, such as oxygen, nitrogen, water, air, hydrogen, and carbon dioxide, allowing gas exchange to occur in the compositions provided herein. In some embodiments, the gas-permeable bag allows for the removal of some of the carbon dioxide present within an interior of the bag by allowing the carbon dioxide to permeate through its wall. In some embodiments, the release of carbon dioxide from the bag can be advantageous to maintaining a desired pH level of the composition contained within the bag.

In some embodiments, the container of the process herein is a gas-permeable container that is closed or sealed. In some embodiments, the container is a container that is closed or sealed and a portion of which is gas-permeable. In some embodiments, the surface area of a gas-permeable portion of a closed or sealed container (e.g., bag) relative to the volume of the product being contained in the container (hereinafter referred to as the “SA/V ratio”) can be adjusted to improve pH maintenance of the compositions provided herein. For example, in some embodiments, the SA/V ratio of the container can be at least about 2.0 cm²/mL (e.g., at least about 2.1 cm²/mL, at least about 2.2 cm²/mL, at least about 2.3 cm²/mL, at least about 2.4 cm²/mL, at least about 2.5 cm²/mL, at least about 2.6 cm²/mL, at least about 2.7 cm²/mL, at least about 2.8 cm²/mL, at least about 2.9 cm²/mL, at least about 3.0 cm²/mL, at least about 3.1 cm²/mL, at least about 3.2 cm²/mL, at least about 3.3 cm²/mL, at least about 3.4 cm²/mL, at least about 3.5 cm²/mL, at least about 3.6 cm²/mL, at least about 3.7 cm²/mL, at least about 3.8 cm²/mL, at least about 3.9 cm²/mL, at least about 4.0 cm²/mL, at least about 4.1 cm²/mL, at least about 4.2 cm²/mL, at least about 4.3 cm²/mL, at least about 4.4 cm²/mL, at least about 4.5 cm²/mL, at least about 4.6 cm²/mL, at least about 4.7 cm²/mL, at least about 4.8 cm²/mL, at least about 4.9 cm²/mL, or at least about 5.0 cm²/mL. In some embodiments, the SA/V ratio of the container can be at most about 10.0 cm²/mL (e.g., at most about 9.9 cm²/mL, at most about 9.8 cm²/mL, at most about 9.7 cm²/mL, at most about 9.6 cm²/mL, at most about 9.5 cm²/mL, at most about 9.4 cm²/mL, at most about 9.3 cm²/mL, at most about 9.2 cm²/mL, at most about 9.1 cm²/mL, at most about 9.0 cm²/mL, at most about 8.9 cm²/mL, at most about 8.8 cm²/mL, at most about 8.7 cm²/mL, at most about 8.6, cm²/mL at most about 8.5 cm²/mL, at most about 8.4 cm²/mL, at most about 8.3 cm²/mL, at most about 8.2 cm²/mL, at most about 8.1 cm²/mL, at most about 8.0 cm²/mL, at most about 7.9 cm²/mL, at most about 7.8 cm²/mL, at most about 7.7 cm²/mL, at most about 7.6 cm²/mL, at most about 7.5 cm²/mL, at most about 7.4 cm²/mL, at most about 7.3 cm²/mL, at most about 7.2 cm²/mL, at most about 7.1 cm²/mL, at most about 6.9 cm²/mL, at most about 6.8 cm²/mL, at most about 6.7 cm²/mL, at most about 6.6 cm²/mL, at most about 6.5 cm²/mL, at most about 6.4 cm²/mL, at most about 6.3 cm²/mL, at most about 6.2 cm²/mL, at most about 6.1 cm²/mL, at most about 6.0 cm²/mL, at most about 5.9 cm²/mL, at most about 5.8 cm²/mL, at most about 5.7 cm²/mL, at most about 5.6 cm²/mL, at most about 5.5 cm²/mL, at most about 5.4 cm²/mL, at most about 5.3 cm²/mL, at most about 5.2 cm²/mL, at most about 5.1 cm²/mL, at most about 5.0 cm²/mL, at most about 4.9 cm²/mL, at most about 4.8 cm²/mL, at most about 4.7 cm²/mL, at most about 4.6 cm²/mL, at most about 4.5 cm²/mL, at most about 4.4 cm²/mL, at most about 4.3 cm²/mL, at most about 4.2 cm²/mL, at most about 4.1 cm²/mL, or at most about 4.0 cm²/mL. In some embodiments, the SA/V ratio of the container can range from about 2.0 to about 10.0 cm²/mL (e.g., from about 2.1 cm²/mL to about 9.9 cm²/mL, from about 2.2 cm²/mL to about 9.8 cm²/mL, from about 2.3 cm²/mL to about 9.7 cm²/mL, from about 2.4 cm²/mL to about 9.6 cm²/mL, from about 2.5 cm²/mL to about 9.5 cm²/mL, from about 2.6 cm²/mL to about 9.4 cm²/mL, from about 2.7 cm²/mL to about 9.3 cm²/mL, from about 2.8 cm²/mL to about 9.2 cm²/mL, from about 2.9 cm²/mL to about 9.1 cm²/mL, from about 3.0 cm²/mL to about 9.0 cm²/mL, from about 3.1 cm²/mL to about 8.9 cm²/mL, from about 3.2 cm²/mL to about 8.8 cm²/mL, from about 3.3 cm²/mL to about 8.7 cm²/mL, from about 3.4 cm²/mL to about 8.6 cm²/mL, from about 3.5 cm²/mL to about 8.5 cm²/mL, from about 3.6 cm²/mL to about 8.4 cm²/mL, from about 3.7 cm²/mL to about 8.3 cm²/mL, from about 3.8 cm²/mL to about 8.2 cm²/mL, from about 3.9 cm²/mL to about 8.1 cm²/mL, from about 4.0 cm²/mL to about 8.0 cm²/mL, from about 4.1 cm²/mL to about 7.9 cm²/mL, from about 4.2 cm²/mL to about 7.8 cm²/mL, from about 4.3 cm²/mL to about 7.7 cm²/mL, from about 4.4 cm²/mL to about 7.6 cm²/mL, from about 4.5 cm²/mL to about 7.5 cm²/mL, from about 4.6 cm²/mL to about 7.4 cm²/mL, from about 4.7 cm²/mL to about 7.3 cm²/mL, from about 4.8 cm²/mL to about 7.2 cm²/mL, from about 4.9 cm²/mL to about 7.1 cm²/mL, from about 5.0 cm²/mL to about 6.9 cm²/mL, from about 5.1 cm²/mL to about 6.8 cm²/mL, from about 5.2 cm²/mL to about 6.7 cm²/mL, from about 5.3 cm²/mL to about 6.6 cm²/mL, from about 5.4 cm²/mL to about 6.5 cm²/mL, from about 5.5 cm²/mL to about 6.4 cm²/mL, from about 5.6 cm²/mL to about 6.3 cm²/mL, from about 5.7 cm²/mL to about 6.2 cm²/mL, or from about 5.8 cm²/mL to about 6.1 cm²/mL.

Gas-permeable closed containers (e.g., bags) or portions thereof can be made of one or more various gas-permeable materials. In some embodiments, the gas-permeable bag can be made of one or more polymers including fluoropolymers (such as polytetrafluoroethylene (PTFE) and perfluoroalkoxy (PFA) polymers), polyolefins (such as low-density polyethylene (LDPE), high-density polyethylene (HDPE)), fluorinated ethylene propylene (FEP), polystyrene, polyvinylchloride (PVC), silicone, and any combinations thereof.

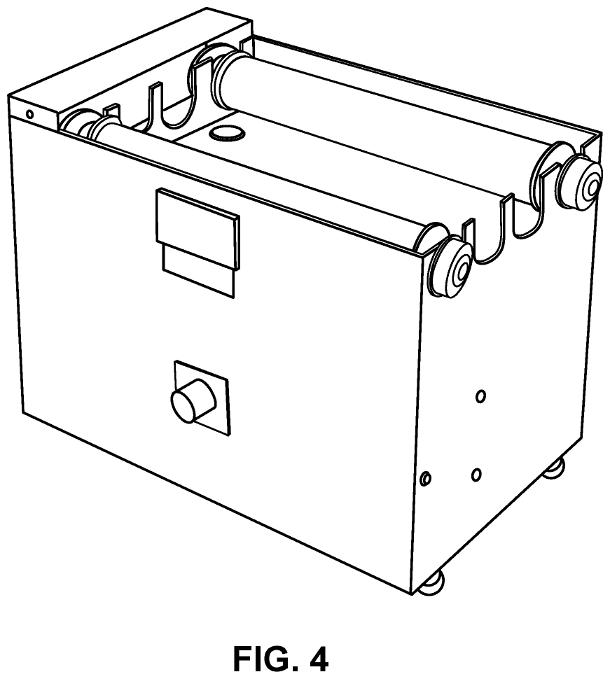

Following drying, the dried plasma can be milled, e.g., into a powder, to form a plasma product. Any appropriate milling method can be used. In some embodiments, a jar mill can be used. For example, a jar mill can be used with about 40% to about 60% (e.g., about 45% to about 55% or about 50%) of the jar volume being product volume and about 10% to 30% (e.g., about 15% to about 25% or about 20%) of the jar volume being grinding media. An exemplary jar mill is shown in FIG. 4. Any appropriate grinding media can be used. A non-limiting example of a grinding media is steel balls. The particle size (e.g., diameter, maximum dimension) of the plasma product can be any appropriate particle size. In some embodiments, the particle size (e.g., diameter, maximum dimension) can be about 0.1 to about 1.0 mm (e.g., about 0.1 to about 0.5 mm or about 0.5 to about 1.0 mm). In some cases, the average particle (e.g., diameter, maximum dimension) size can be about 0.1 to about 1.0 mm (e.g., about 0.1 to about 0.5 mm or about 0.5 to about 1.0 mm). In some cases the mass-median particle size (e.g., diameter, maximum dimension) can be about 0.1 to about 1.0 mm (e.g., about 0.1 to about 0.5 mm or about 0.5 to about 1.0 mm). In some cases, the abundance-median particle size (e.g., diameter, maximum dimension) (e.g., the value of the particle diameter (or maximum dimension) at 50% in the cumulative distribution) can be about 0.1 to about 1.0 mm (e.g., about 0.1 to about 0.5 mm or about 0.5 to about 1.0 mm). Particle size (e.g., diameter, maximum dimension) can be measured using any appropriate method, for example, light microscopy or dynamic light scattering (DLS; e.g., dry DLS). For example, in some embodiments, the particle size (e.g., diameter, maximum dimension) can be about 0.1 to about 1.0 mm (e.g., about 0.1 to about 0.5 mm or about 0.5 to about 1.0 mm) as determined by light microscopy. For example, in some embodiments, the mass-median particle size (e.g., diameter, maximum dimension) can be about 0.1 to about 1.0 mm (e.g., about 0.1 to about 0.5 mm or about 0.5 to about 1.0 mm) as determined by light microscopy. For example, in some embodiments, the abundance-median particle size (e.g., diameter, maximum dimension) (e.g., the value of the particle diameter (or maximum dimension) at 50% in the cumulative distribution) can be about 0.1 to about 1.0 mm (e.g., about 0.1 to about 0.5 mm or about 0.5 to about 1.0 mm) as determined by light microscopy.

The plasma product can be packaged into any appropriate container. In some embodiments, packaging can take place aseptically. In some embodiments, sterilization by filtration can be used. A non-limiting example of an appropriate container is a sterile Freezing Ethyl Vinyl Acetate (EVA) bag (e.g., Macopharma Biotech product GSR7003AU). For example, a plasma product (e.g., any of the plasma products described herein) can be dispensed into the bag, which is then heat sealed, and placed into a moisture barrier bag (e.g., Uline product S-15503) with a desiccant package (e.g., Uline product S-5165). The open ended vinyl bags may be sourced any appropriate supplier.

A plasma product as described herein can be stored under any appropriate conditions. For example, a plasma product as described herein can be stored at room temperature (e.g., about 18 to about 30° C.) (e.g., in the dark).

In some embodiments, the plasma products provided herein may be stable for extended periods of time. In some embodiments, a plasma product as described herein can be is stable for at least three months (e.g., at least six months, at least one year, or at least two years) at a temperature of about 15° F. to about 140° F. (e.g., about 15° F. to about 30° F., about 15° F. to about 50° F., about 15° F. to about 75° F., about 15° F. to about 100° F., about 15° F. to about 125° F., about 30° F. to about 140° F., about 50° F. to about 140° F., about 75° F. to about 140° F., about 100° F. to about 140° F., or about 125° F. to about 140° F.). In some embodiments, a plasma product as described herein can be stable for at least three months (e.g., at least six months, at least one year, or at least two years) at a temperature of about −9.5° C. to about 60° C. (e.g., about −9.5° C. to about 0° C., about −9.5° C. to about 10° C., about −9.5° C. to about 20° C., about −9.5° C. to about 30° C., about −9.5° C. to about 40° C., about −9.5° C. to about 50° C., about 0° C. to about 60° C., about 10° C. to about 60° C., about 20° C. to about 60° C., about 30° C. to about 60° C., about 40° C. to about 60° C., or about 50° C. to about 60° C.). Stability can be determined by any appropriate method. Non-limiting examples of parameters that can be used to determine stability include coagulant activity of coagulation factors (e.g., Factor VII, Factor VIII), base clotting times (e.g., Prothrombin time (PT) or activated partial thromboplastin time (APTT)), residual moisture (e.g., using Karl Fischer titration), von Willebrand Factor antigen percentage, fibrinogen content, albumin content, pH, and package integrity. In some embodiments, a plasma product is stable if one or more of coagulant activity of Factor VII, coagulant activity of Factor VIII, PT base clotting time, APTT PT base clotting time, residual moisture, von Willebrand Factor antigen percentage, fibrinogen content, albumin content, pH, or package integrity vary by 30% or less, such as 20% or less such as 10% or less over a period of at least three months (e.g., at least six months, at least one year, or at least two years) at a temperature of about −9.5° C. to about 60° C. (e.g., about −9.5° C. to about 0° C., about −9.5° C. to about 10° C., about −9.5° C. to about 20° C., about −9.5° C. to about 30° C., about −9.5° C. to about 40° C., about −9.5° C. to about 50° C., about 0° C. to about 60° C., about 10° C. to about 60° C., about 20° C. to about 60° C., about 30° C. to about 60° C., about 40° C. to about 60° C., or about 50° C. to about 60° C.).

A plasma product can be rehydrated to form a rehydrated plasma product. A rehydrated plasma product can be used, e.g., for administration to a subject (e.g., via transfusion). A plasma product can be rehydrated with any appropriate solution, for example water (e.g., sterile water for injection) or a buffer. Any appropriate rehydration volume or mass can be used. In some embodiments, a volume that is about the same volume of the acidified plasma prior to drying is used rehydrate the plasma product (for example, if a plasma product was generated from 250 mL of plasma, about 250 mL of a solution can be used to rehydrate it), though this ratio can vary. In some cases, the solid content of a plasma product (e.g., produced as described herein) is about 6.5% by mass of the mass of the acidified plasma used to make it. Accordingly, in some embodiments, a rehydration mass can be used to reach about 100% of the mass of the acidified plasma, though the ratio can vary. For example, a rehydration mass of about 10-fold to about 20-fold (e.g., about 12-fold to about 18-fold or about 14-fold to about 16-fold) the mass of the plasma product can be used. In some embodiments, a rehydration mass of about 14.0-fold to about 14.5-fold the mass of the plasma product can be used. In some embodiments, rehydrating can include rehydrating over a period of less than 3 minutes (e.g., less than 2 minutes or less than 1 minute). In some embodiments, rehydrating can include rehydrating over a period of about 1 to about 3 minutes (e.g., about 1 to about 2 minutes).

In some embodiments, a rehydrated plasma product can be used immediately. In some embodiments, a rehydrated plasma product can be refrigerated for up to about 5 days (e.g., at about 4 to about 8° C.).

A rehydrated plasma product can have any appropriate properties. For example, a rehydrated plasma product can have a fibrinogen concentration of at least about 150 mg/dL (e.g., at least about 175, 200, 225, 250, 275, or 300 mg/dL). A fibrinogen concentration can be measured using any appropriate method. As another example, a rehydrated plasma product can have a von Willebrand Factor antigen percentage of at least about 50% (e.g., at least about 60%, 70%, 80%, 90%, or 100%). A von Willebrand Factor antigen percentage can be measured using any appropriate method. In some embodiments, a rehydrated plasma product can have a Factor VII coagulant activity (FVII:C) of at least about 30% (e.g., at least about 50%, 70%, 90% or 100%). A Factor VII coagulant activity can be measured using any appropriate method. In some embodiments, a rehydrated plasma product can have a Factor VIII coagulant activity (FVIII:C) of at least about 50% (e.g., at least about 70%, 90%, 100%, 110%, 120%, 140%, or 160%). A Factor VIII coagulant activity can be measured using any appropriate method. In some embodiments, a rehydrated plasma product can have an albumin concentration of at least about 1.5 mg/dL (e.g., at least about 1.8, 2.0, 2.2, 2.4, 2.6, 2.8, 3.0, or 3.2 mg/dL). An albumin concentration can be measured using any appropriate method. In some embodiments, a rehydrated plasma product can have a pH of at least about 5.5 (e.g., at least about 6.0, 6.5, or 7.0). In some embodiments, a rehydrated plasma product can have a pH of between about 5.5 and about 7.8 (e.g., between about 6.0 and about 7.8, about 6.5 and about 7.8, about 7.0 and about 7.8, or about 7.2 and about 7.6).

Also provided herein are plasma products produced by any of the methods of the disclosure.

Also provided herein are rehydrated plasma products produced by any of the methods of the disclosure.

Also provided herein are methods of administering one or more of the products described herein. In some embodiments, the method can include providing a plasma product as described herein, rehydrating the plasma product (e.g., as described herein) to form a rehydrated plasma product, and administering the rehydrated plasma product to a subject. In some embodiments, the method can include providing a rehydrated plasma product as described herein and administering the rehydrated plasma product to a subject. Administering can include any appropriate steps. For example, administering can include transfusing. As another example, administering can include intravenous administration. In some embodiments, administering can include intraosseous administration. Administration can include filtering through a filter with a pore size of about 170 to about 260 microns before the rehydrated plasma product enters the subject (e.g., through an inline filter). Administration can include using an automated fluid pump or syringe pump. In some embodiments, administering a product as described herein can exclude the administration of other fluids. A subject can be any appropriate subject. For example, a subject can be a canine subject. In some embodiments, a subject can be a feline subject. In some embodiments, a subject can be an equine subject. In some embodiments, a subject can be a porcine subject. In some embodiments, a subject can be a bovine subject. In some embodiments, a subject can be a simian subject. In some embodiments, a subject can be a human subject. In some embodiments of the methods described herein, the subject can have undergone a trauma. In some embodiments of the methods described herein, the subject can have a coagulopathy (e.g., acquired or congenital). In some embodiments, the method does not include administering platelets or platelet derivative to the subject. In some embodiments, the method does not include administering red blood cells (and/or red blood cell substitutes) to the subject.

Exemplary prescribing information for an exemplary plasma product as described herein is shown in the Appendix to this disclosure. Exemplary prescribing information is also shown below:

DESCRIPTION: StablePlas RX™ is a hemostatically active, lyophilized plasma generated from allogeneic pooled canine fresh frozen plasma for rapid delivery in canine trauma resuscitation and coagulopathy management. The product is available as a sterile non-pyrogenic white to tan freeze-dried powder that when reconstituted according to label instructions results in a clear to light gold liquid. Rehydrated material contains albumin, plasma proteins, coagulation factors, and immunoglobulins consistent with materials found in canine fresh frozen plasma.

INDICATION: An intravenous administration developed for the treatment of coagulopathy and trauma resuscitation in canine patients.

DOSAGE AND ADMINISTRATION: StablePlas is for intravenous administration. The dose is 3 ml of reconstituted solution per kilogram of body weight for trauma resuscitation. In treatment of coagulopathy (acquired or congenital), a dose of 10-15 ml per kilogram of body weight is recommended. The product is administered by a single intravenous infusion using a standard macroaggregate filter (170-260 microns) by intravenous or intraosseous route.

Rehydration Instructions:
60 ml bag: Insert the supplied sterile water (56 ml) for injection (WFI) into the rehydration port of the bag, marked with a blue port. Mix the WFI into the plasma bag using a slow swirling motion. Do NOT allow it to FOAM.
120 ml bag: Insert the supplied sterile water (112 ml) for injection (WFI) into the rehydration port on the bag, marked with a blue port. Mix the WFI into the plasma bag using a slow motion. Do NOT allow it to FOAM.
240 ml bag: Insert the supplied sterile water (224 ml) for injection (WFI) into the rehydration port on the bag, marked with a blue port. Mix WFI into the plasma bag using a slow swirling motion. Do NOT allow it to FOAM.
Ensure the product does not have any large particles or aggregates of powder; if present swirl again and allow to sit for 5 minutes.

Dosing Instructions:

Break the seal on the infusion line and allow the line to fill with plasma.

Administer according to the standard of care for the disease indication.

Do not mix with other products or solutions.

Use only the in-line provided blood filter for administration.

Flush catheter with an appropriate amount of saline after administration.

This product may be administered by automated fluid pump or syringe pump.

CONTRAINDICATIONS: Do not administer concurrently with other fluids.

WARNINGS: Not for use in Humans. Keep out of reach of children. Only to be administered to canine patients under the care of a licensed veterinarian.

PRECAUTIONS: The safe use of StablePlas has not been evaluated for use in animals under 9 months of age or during pregnancy.

ADVERSE REACTIONS: Good Laboratory Practice (GLP) acute toxicity study in canines have found no definitive product related toxicities. For technical assistance or to report suspected adverse reactions to StablePlas, contact the manufacturer. For additional information about adverse drug experience reporting animal drugs, contact FDA at 1-888-FDA-VETS or fda.gov/AnimalVeterinary/SafetyHealth

SAFETY AND EFFECTIVENESS SUMMARY: In a safety study utilizing an automated plasma exchange model in canines with a 1.2×plasma volume exchange, six healthy mongrels were randomized to receive either StablePlas or pooled FFP. Each dog underwent automated plasma exchange with a Hemonetics MCS+apheresis device. No adverse reactions were associated with the administration StablePlas. One dog in the FFP group experienced signs associated with anaphylaxis (diarrhea, facial edema). No difference were noted in CBC, biochemistry profile, TEG, PT, aPTT, ionized calcium, venous pH or lactate between groups. An abstract was presented at Veterinary Trauma and Critical Care/VETCOT 2019 (Mays, E. L., Hale, A., Montgomery, J. & Fitzpatrick, G. M. (2019). Lyophylized Plasma (StablePlas™) is safe and noninferior to fresh frozen plasma during plasma exchange in the dog. Veterinary Trauma and Critical Care/VETCOT).

STORAGE CONDITION: Unopened, in-date bags should be stored at room temperature (18 to 30° C.) within the foil outer pouch. This product should be utilized immediately after rehydration or refrigerated for up to 5 days (4-8° C.). Unused product should be disposed of as clinical biological waste.

How Supplied:
60 ml bag, in a single carton.
120 ml bag, in a single carton.
240 ml bag, in a single carton.

In some embodiments, a subject can be administered about 3 mL of a rehydrated plasma product per kilogram of the subject, for example, if the subject has undergone trauma. In some embodiments, a subject can be administered about 10 to about 15 mL of a rehydrated plasma product per kilogram of the subject, for example, if the subject has a coagulopathy.

The present invention is not limited to that precisely as shown and described. Specific embodiments disclosed herein may be further limited in the claims using “consisting of” or “consisting essentially of” language. Embodiments of the invention so claimed are inherently or expressly described and enabled herein.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein.

### EXEMPLARY EMBODIMENTS

Embodiment 1 is a method of preparing a plasma product, the method comprising:
providing plasma;
acidifying the plasma to form acidified plasma;
drying the acidified plasma to form dried plasma; and
milling the dried plasma to form a plasma product.

Embodiment 2 is a method of preparing a plasma product, the method comprising:
acidifying plasma to form acidified plasma;
drying the acidified plasma to form dried plasma; and
milling the dried plasma to form a plasma product.

Embodiment 3 is a method of preparing a plasma product, the method consisting essentially of:
providing plasma;
acidifying the plasma to form acidified plasma;
drying the acidified plasma to form dried plasma; and
milling the dried plasma to form a plasma product.

Embodiment 4 is a method of preparing a plasma product, the method consisting essentially of:
acidifying plasma to form acidified plasma;
drying the acidified plasma to form dried plasma; and
milling the dried plasma to form a plasma product.

Embodiment 5 is a method of preparing a plasma product, the method consisting of:
providing plasma;
acidifying the plasma to form acidified plasma;
drying the acidified plasma to form dried plasma; and
milling the dried plasma to form a plasma product.

Embodiment 6 is a method of preparing a plasma product, the method consisting of:
acidifying plasma to form acidified plasma;
drying the acidified plasma to form dried plasma; and
milling the dried plasma to form a plasma product.

Embodiment 7 is the method of any one of embodiments 1-6, wherein the plasma is canine plasma.

Embodiment 8 is the method of any one of embodiments 1-6, wherein the plasma is feline plasma.

Embodiment 9 is the method of any one of embodiments 1-6, wherein the plasma is equine plasma.

Embodiment 10 is the method of any one of embodiments 1-6, wherein the plasma is bovine plasma.

Embodiment 11 is the method of any one of embodiments 1-6, wherein the plasma is porcine plasma.

Embodiment 12 is the method of any one of embodiments 1-6, wherein the plasma is simian plasma.

Embodiment 13 is the method of any one of embodiments 1-6, wherein the plasma is human plasma.

Embodiment 14 is the method of any one of embodiments 1-13, wherein the plasma is pooled plasma.

Embodiment 15 is the method of any one of embodiments 1-14, wherein acidifying the plasma comprises contacting the plasma with about 0.5% to about 2.5% (v/v) of a 1N acid.

Embodiment 16 is the method of any one of embodiments 1-15, wherein acidifying the plasma comprises contacting the plasma with about 1.25% to about 1.7% (v/v) of a 1N acid.

Embodiment 17 is the method of embodiment 15 or embodiment 16, wherein the acid is a mineral acid.

Embodiment 18 is the method of embodiment 17, wherein the acid is hydrochloric acid.

Embodiment 19 is the method of any one of embodiments 1-18, wherein the method does not comprise addition of platelets or platelet derivatives to the plasma, the acidified plasma, the dried plasma, or the plasma product.

Embodiment 20 is the method of any one of embodiments 1-19, wherein the method does not comprise addition of red blood cells to the plasma, the acidified plasma, the dried plasma, or the plasma product.

Embodiment 21 is the method of any one of embodiments 1-20, wherein the plasma does not comprise an exogenous polymer.

Embodiment 22 is the method of any one of embodiments 1-21, wherein the plasma does not comprise an exogenous saccharide.

Embodiment 23 is the method of any one of embodiments 1-22, wherein the method does not comprise addition of components other than acid to the plasma.

Embodiment 24 is the method of any one of embodiments 1-23, wherein drying the acidified plasma comprises lyophilizing the acidified plasma.

Embodiment 25 is the method of embodiment 24, wherein lyophilizing comprises using the lyophilization protocol in Table 1.

Embodiment 26 is the method of embodiment 24, wherein lyophilizing comprises:
freezing the acidified plasma to below −5° C. to form frozen plasma;
reducing the pressure to less than 100 mTorr;
drying the frozen plasma to form dried plasma.

Embodiment 27 is the method of embodiment 26, wherein drying the frozen plasma occurs at atmospheric pressure.

Embodiment 28 is the method of embodiment 26 or embodiment 27, wherein drying the frozen plasma comprises a starting temperature of below 0° C.

Embodiment 29 is the method of embodiment 28, wherein the starting temperature is about −5° C.

Embodiment 30 is the method of any one of embodiments 26-29, wherein drying the frozen plasma comprises an ending temperature above 0° C.

Embodiment 31 is the method of embodiment 26, wherein the ending temperature is about 25° C.

Embodiment 32 is the method of any one of embodiments 26-31, wherein the freezing step has a duration of at least about 1 hour.

Embodiment 33 is the method of any one of embodiments 26-31, wherein the drying step has a duration of at least 24 hours.

Embodiment 34 is the method of any one of embodiments 26-31, wherein the drying step has a duration of at least 48 hours.

Embodiment 35 is the method of any one of embodiments 26-31, wherein the drying step has a duration of at least 72 hours.

Embodiment 36 is the method of any one of embodiments 26-31, wherein the drying step comprises a primary drying step and a secondary drying step.

Embodiment 37 is the method of any one of embodiments 1-23, wherein drying the acidified plasma comprises spray drying the acidified plasma.

Embodiment 38 is the method of any one of embodiments 1-37, wherein the method further comprises rehydrating the plasma product to form a rehydrated plasma product.

Embodiment 39 is the method of embodiment 38, wherein rehydrating comprises rehydrating with water.

Embodiment 40 is the method of embodiment 38, wherein rehydrating comprises rehydrating with a buffer.

Embodiment 41 is the method of any one of embodiments 38-40, wherein rehydrating the plasma product comprises contacting the plasma product with a mass of water or buffer about 10-fold to about 20-fold of the mass of the plasma product.

Embodiment 42 is the method of any one of embodiments 38-40, wherein rehydrating the plasma product comprises contacting the plasma product with a mass of water or buffer about 12-fold to about 18-fold of the mass of the plasma product.

Embodiment 43 is the method of any one of embodiments 38-40, wherein rehydrating the plasma product comprises contacting the plasma product with a mass of water or buffer about 14-fold to about 16-fold of the mass of the plasma product.

Embodiment 44 is the method of any one of embodiments 38-43, wherein the rehydrated plasma product has a fibrinogen concentration of at least about 150 mg/dL.

Embodiment 45 is the method of any one of embodiments 38-43, wherein the rehydrated plasma product has a fibrinogen concentration of at least about 200 mg/dL.

Embodiment 46 is the method of any one of embodiments 38-43, wherein the rehydrated plasma product has a fibrinogen concentration of at least about 250 mg/dL.

Embodiment 47 is the method of any one of embodiments 38-46, wherein the rehydrated plasma product has a von Willebrand Factor antigen percentage of at least about 50%.

Embodiment 48 is the method of any one of embodiments 38-46, wherein the rehydrated plasma product has a von Willebrand Factor antigen percentage of at least about 70%.

Embodiment 49 is the method of any one of embodiments 38-46, wherein the rehydrated plasma product has a von Willebrand Factor antigen percentage of at least about 100%.

Embodiment 50 is the method of any one of embodiments 38-49, wherein the rehydrated plasma product has a Factor VII coagulant activity of at least about 30%.

Embodiment 51 is the method of any one of embodiments 38-49, wherein the rehydrated plasma product has a Factor VII coagulant activity of at least about 50%.

Embodiment 52 is the method of any one of embodiments 38-49, wherein the rehydrated plasma product has a Factor VII coagulant activity of at least about 100%.

Embodiment 53 is the method of any one of embodiments 38-52, wherein the rehydrated plasma product has a Factor VIII coagulant activity of at least about 50%.

Embodiment 54 is the method of any one of embodiments 38-52, wherein the rehydrated plasma product has a Factor VIII coagulant activity of at least about 70%.

Embodiment 55 is the method of any one of embodiments 38-52, wherein the rehydrated plasma product has a Factor VIII coagulant activity of at least about 100%.

Embodiment 56 is the method of any one of embodiments 38-55, wherein the rehydrated plasma product has an albumin concentration of at least about 1.5 mg/dL.

Embodiment 57 is the method of any one of embodiments 38-55, wherein the rehydrated plasma product has an albumin concentration of at least about 2.2 mg/dL.

Embodiment 58 is the method of any one of embodiments 38-55, wherein the rehydrated plasma product has an albumin concentration of at least about 2.5 mg/dL.

Embodiment 59 is the method of any one of embodiments 38-58, wherein the rehydrated plasma product has a pH of at least about 5.5.

Embodiment 60 is the method of any one of embodiments 38-59, wherein the rehydrated plasma product has a pH between about 5.5 and about 7.8.

Embodiment 61 is the method of any one of embodiments 38-60, wherein the rehydrated plasma product has a pH of at least about 6.0.

Embodiment 62 is the method of any one of embodiments 38-61, wherein the rehydrated plasma product has a pH of between about 6.0 and about 7.8.

Embodiment 63 is the method of any one of embodiments 38-62, wherein the rehydrated plasma product has a pH of at least about 6.5.

Embodiment 64 is the method of any one of embodiments 38-63, wherein the rehydrated plasma product has a pH of between about 6.5 and about 7.8.

Embodiment 65 is the method of any one of embodiments 38-64, wherein the rehydrated plasma product has a pH of about 7.0 to about 7.8.

Embodiment 66 is the method of any one of embodiments 38-65, wherein the rehydrated plasma product has a pH of about 7.2 to about 7.6.

Embodiment 67 is the method of any one of embodiments 38-66, wherein rehydrating comprises rehydrating over a period of between 1 and 3 minutes.

Embodiment 68 is the method of any one of embodiments 38-66, wherein rehydrating comprises rehydrating over a period of less than 2 minutes.

Embodiment 69 is the method of any one of embodiments 38-66, wherein rehydrating comprises rehydrating over a period of less than 1 minute.

Embodiment 70 is the method of any one of embodiments 38-66, wherein rehydrating comprises rehydrating over a period of 1-2 minutes.

Embodiment 71 is the method of any one of embodiments 1-37, wherein the plasma product is stable for at least two years at a temperature of about 15° F. to about 140° F.

Embodiment 72 is the method of any one of embodiments 1-37, wherein the plasma product is stable for at least two years at a temperature of about −9.5° C. to about 60° C.

Embodiment 73 is a plasma product produced by the method of any one of any one of embodiments 1-37.

Embodiment 74 is a rehydrated plasma product produced by the method of any one of embodiments 38-70.

Embodiment 75 is a method of treating a subject, the method comprising:

rehydrating a plasma product produced by the method of any one of embodiments 1-37 to form a rehydrated plasma product; and

administering the rehydrated plasma product to the subject.

Embodiment 76 is a method of treating a subject, the method comprising:

administering a rehydrated plasma product produced by the method of any one of embodiments 37-70 to the subject.

Embodiment 77 is the method of any one of embodiments 75-76, wherein the subject has undergone trauma.

Embodiment 78 is the method of any one of embodiments 75-76, wherein the subject has a coagulopathy.

Embodiment 79 is the method of any one of embodiments 75-78, wherein the subject is a canine subject.

Embodiment 80 is the method of any one of embodiments 75-78, wherein the subject is a feline subject.

Embodiment 81 is the method of any one of embodiments 75-78, wherein the subject is an equine subject.

Embodiment 82 is the method of any one of embodiments 75-78, wherein the subject is a bovine subject.

Embodiment 83 is the method of any one of embodiments 75-78, wherein the subject is a porcine subject.

Embodiment 84 is the method of any one of embodiments 75-78, wherein the subject is a simian subject.

Embodiment 85 is the method of any one of embodiments 75-78, wherein the subject is a human subject.

Embodiment 86 is the method of any one of embodiments 75-85, wherein the method does not comprise administering platelets or platelet derivative to the subject.

Embodiment 87 is the method of any one of embodiments 75-86, wherein the method does not comprise administering red blood cells to the subject.

### EXAMPLES

### Example 1

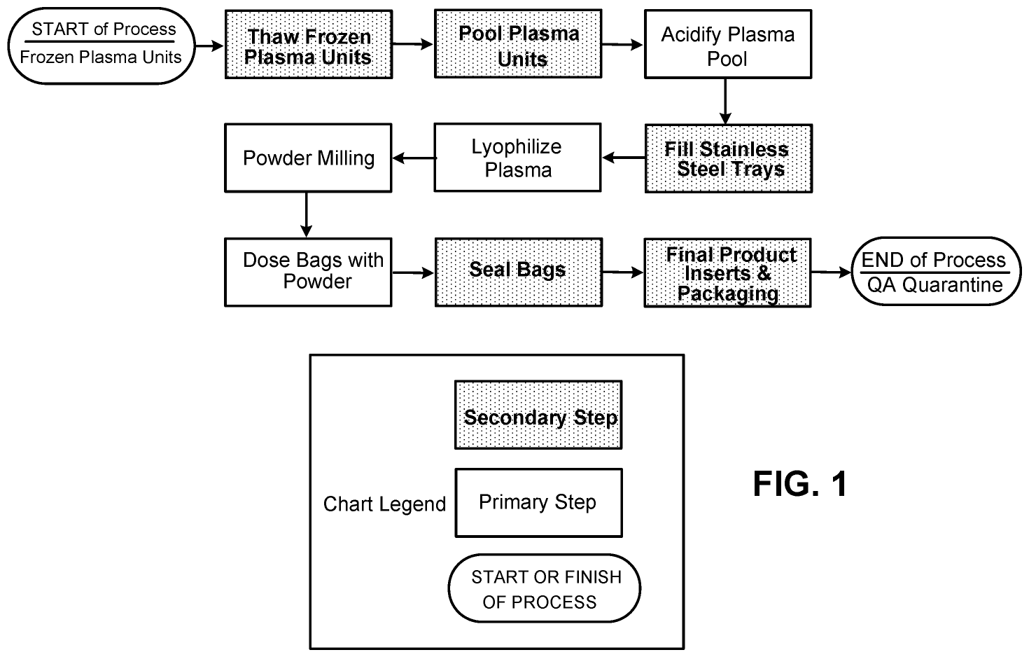

A lyophilized plasma product was prepared generally as shown in FIG. 1. Briefly, frozen canine plasma units were thawed and pooled. The pooled plasma was acidified with various amounts of hydrochloric acid.

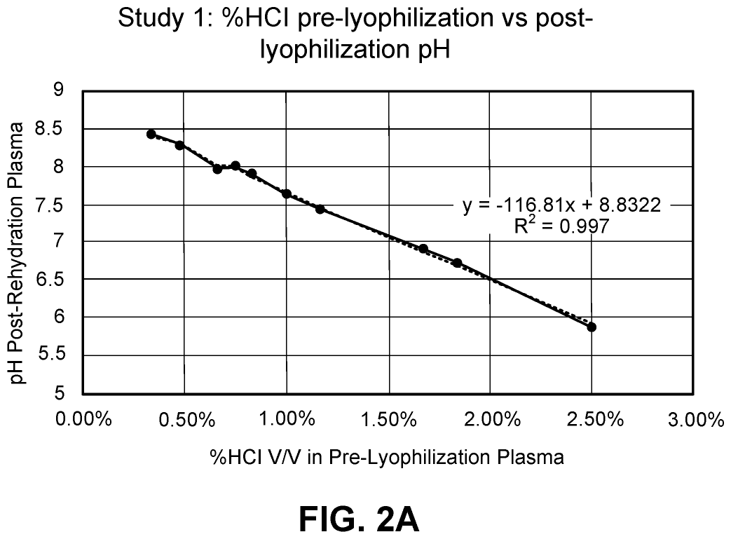

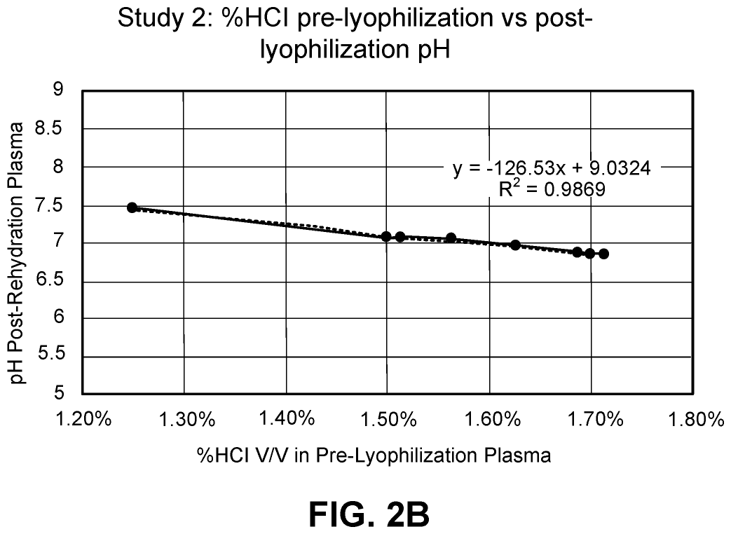

The amount of hydrochloric acid (HCl) used can have an effect on the final pH of the rehydrated plasma. Using 1N HCl, concentrations (% v/v) of about 0.5% to about 2.5% were determined to result in a pH of the rehydrated plasma of about 5.8 to about 8.5 (FIG. 2A). A closer study of about 1.25% to about 1.7% HCl showed that this range resulted in rehydrated plasma with a pH of about 6.8 to about 7.5 (FIG. 2B).

### Example 2

Acidified pooled canine plasma was prepared as in Example 1, then lyophilized (Table 1) and milled into a powder.

TABLE 1
Lyophilization protocol

| | Phase | Temperature | | | Vacuum |
|---|---|---|---|---|---|
| Phase | Type | Setpoint | | Time | Setpoint |
| Freeze | Hold | −50 | C. | 300 minutes | N/A |
| Vacuum | Hold | −50 | C. | Variable | <100 mTorr |
| Primary | Ramp | −5 | C. | 540 minutes | 0 mTorr |
| Dry | Hold | −5 | C. | 1080 minutes | 0 mTorr |
| | Ramp | +5 | C. | 120 minutes | 0 mTorr |
| | Hold | +5 | C. | 2040 minutes | 0 mTorr |
| Secondary | Ramp | +25 | C. | 240 minutes | 0 mTorr |
| Dry | Hold | +25 | C. | 240 minutes | 0 mTorr |
| | Hold | +25 | C. | >60 minutes | 0 mTorr |

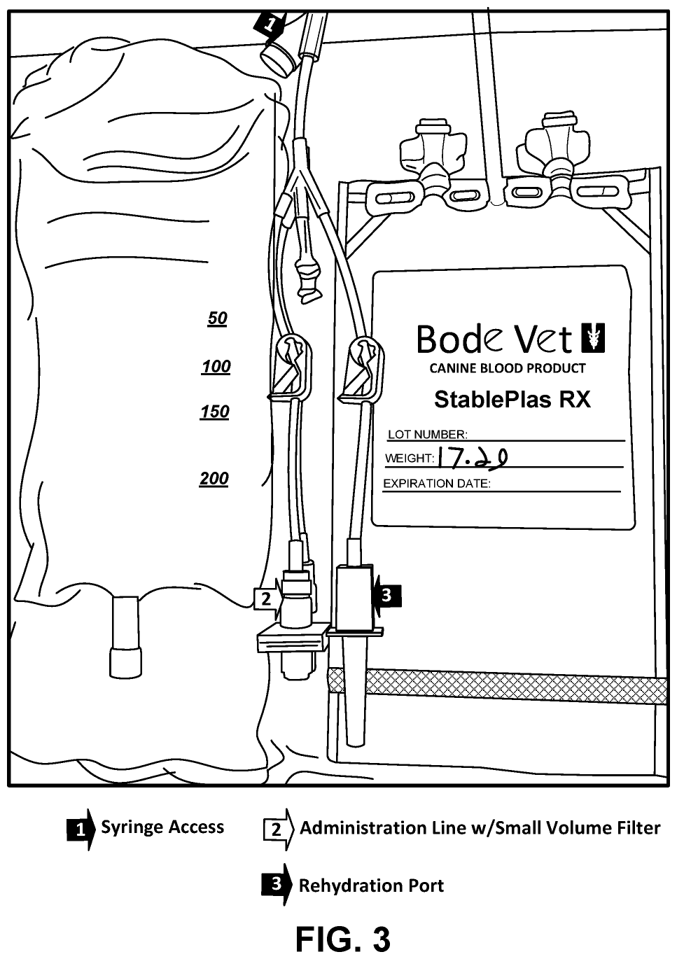

The milled powder was filled into bags and sealed. The bags can be further labeled and packaged (an example labeled product is shown in FIG. 3), and optionally quarantined.

### Example 3

Acidified pooled canine plasma was prepared as in Example 1, then lyophilized and milled into a powder as in Example 2, then rehydrated. Properties of the rehydrated plasma were evaluated. Threshold and objective values for these properties are shown in Table 2, while measured values are shown in Table 3. In these tables, “Fib” is fibrinogen, “VWF:Ag” is Von Willebrand Factor antigen, “FVII:C” is Factor VII procoagulant activity, and “FVIII:C” is Factor VIII procoagulant activity.

TABLE 2
Threshold and objective property values.
Dried Canine Plasma - Target Product Profile

| | Fib | VWF:Ag | FVII:C | FVIII:C | Albumin | | Rehydration |
|---|---|---|---|---|---|---|---|
| | (mg/dL) | (%) | (%) | (%) | (mg/dL) | pH | Time (min) |
| Threshold | >150 | ≥50 | ≥30 | ≥50 | >1.5 | >5.5 | 2-3 |
| Objective | >200 | ≥70 | ≥50 | ≥70 | >2.2 | 7.4 | <2 |

TABLE 3
Measured property values.

| | Bulk Lyophilization Dried Canine Plasma - Actual Values | | | | | | |
|---|---|---|---|---|---|---|---|
| | | | | | | | Rehyd. |
| | Fib | VWF:Ag | FVII:C | FVIII:C | Albumin | | Time |
| | (mg/dL) | (%) | (%) | (%) | (mg/dL) | pH | (min) |
| Lyophilized Plasma | 274 | 104 | 95 | 145 | 2.8 | 6.73 | 0.6 |
| (adjusted to ~pH 6.5) | | | | | | | |
| [Regular Rehy.] | | | | | | | |
| Lyophilized Plasma | 353 | 122 | 105 | 164 | 3.4 | 6.73 | 0.6 |
| (adjusted to ~pH 6.5) | | | | | | | |
| [5% less water] | | | | | | | |
| Lyophilized Plasma | 320 | 122 | 105 | 144 | 3.1 | 6.08 | 0.7 |
| (adjusted to ~pH 6.0) | | | | | | | |
| [Regular Rehy.] | | | | | | | |
| Lyophilized Plasma | 305 | 122 | 107 | 133 | 2.9 | 6.08 | 0.7 |
| (adjusted to ~pH 6.0) | | | | | | | |
| [5% less water] | | | | | | | |

### Example 3

The long term stability of products prepared by the method of Example 2 was tested at 25° C. and 75% relative humidity (RH) using two batches. The batches were characterized by fibrinogen content (Table 4), coagulation factor activity (factors VII (Table 5) and VIII (Table 6)), von Willebrand Factor activity (Table 7), Albumin content (Table 8), and pH (Table 9). In each Table, ND=not determined. After 6 months of storage, the product still met all acceptance criteria and did not demonstrate any significant evolution in product quality.

TABLE 4

| | Fibrinogen (mg/dL) | | |
|---|---|---|---|
| Month | Spec | Batch A | Batch B |
| 0 | 150 | 202 | 219 |
| 0 | 150 | 204 | 198 |
| 0 | 150 | 198 | 207 |
| 1 | 150 | ND | 189 |
| 3 | 150 | 186 | 180 |
| 3 | 150 | 182 | 192 |
| 6 | 150 | 184 | ND |
| 6 | 150 | 191 | ND |

TABLE 5

| | FVII:C (%) | | |
|---|---|---|---|
| Month | Spec | Batch A | Batch B |
| 0 | 30 | 62 | 57 |
| 0 | 30 | 38 | 51 |
| 0 | 30 | 52 | 52 |
| 1 | 30 | ND | 53 |
| 3 | 30 | 41 | 50 |
| 3 | 30 | 40 | 52 |
| 6 | 30 | 42 | ND |
| 6 | 30 | 44 | ND |

TABLE 6

| | FVIII:C (%) | | |
|---|---|---|---|
| Month | Spec | Batch A | Batch B |
| 0 | 50 | 105 | 147 |
| 0 | 50 | 114 | 128 |
| 0 | 50 | 107 | 133 |
| 1 | 50 | ND | 88 |
| 3 | 50 | 69 | 64 |
| 3 | 50 | 70 | 69 |
| 6 | 50 | 57 | ND |
| 6 | 50 | 64 | ND |

TABLE 7

| | VWF:Ag(%) | | |
|---|---|---|---|
| Month | Spec | Batch A | Batch B |
| 0 | 50 | 107 | 109 |
| 0 | 50 | 102 | 107 |
| 0 | 50 | 100 | 106 |
| 1 | 50 | ND | 98 |
| 3 | 50 | 81 | 69 |
| 3 | 50 | 82 | 74 |
| 6 | 50 | 74 | ND |
| 6 | 50 | 84 | ND |

TABLE 8

| | Albumin (g/dL) | | |
|---|---|---|---|
| Month | Spec | Batch A | Batch B |
| 0 | 1.5 | 2.4 | 2.6 |
| 0 | 1.5 | 2.4 | 2.5 |
| 0 | 1.5 | 2.4 | 2.5 |
| 1 | 1.5 | ND | 2.7 |
| 3 | 1.5 | 2.5 | 2.4 |
| 3 | 1.5 | 2.4 | 2.6 |
| 6 | 1.5 | 2.6 | ND |
| 6 | 1.5 | 2.5 | ND |

TABLE 9

| | pH | | |
|---|---|---|---|
| Month | Spec | Batch A | Batch B |
| 0 | 5.5 | 7.4 | 7.31 |
| 0 | 5.5 | 7.4 | 7.31 |
| 0 | 5.5 | 7.4 | 7.31 |
| 1 | 5.5 | ND | 7.32 |
| 3 | 5.5 | 7.26 | 7.16 |
| 3 | 5.5 | ND | ND |
| 6 | 5.5 | 7.01 | ND |
| 6 | 5.5 | ND | ND |

1. (canceled)

2. A method of preparing a plasma product, the method comprising:
acidifying plasma to form acidified plasma;
drying the acidified plasma to form dried plasma; and
milling the dried plasma to form a plasma product.

3. The method of claim 2, wherein the plasma is canine plasma.

4. The method of claim 2, wherein the plasma is human plasma.

5. (canceled)

6. The method of claim 2, wherein acidifying the plasma comprises contacting the plasma with about 0.5% to about 2.5% (v/v) of a 1N acid.

7. (canceled)

8. (canceled)

9. (canceled)

10. (canceled)

11. (canceled)

12. The method of claim 2, wherein the method further comprises rehydrating the plasma product to form a rehydrated plasma product.

13. (canceled)

14. (canceled)

15. The method of claim 12, wherein rehydrating the plasma product comprises contacting the plasma product with a mass of water or buffer about 10-fold to about 20-fold of the mass of the plasma product.

16. The method of claim 12, wherein the rehydrated plasma product has a fibrinogen concentration of at least about 150 mg/dL.

17. The method of claim 12, wherein the rehydrated plasma product has a von Willebrand Factor antigen percentage of at least about 50%.

18. The method of claim 12, wherein the rehydrated plasma product has a Factor VII coagulant activity of at least about 30%.

19. The method of claim 12, wherein the rehydrated plasma product has a Factor VIII coagulant activity of at least about 50%.

20. The method of claim 12, wherein the rehydrated plasma product has an albumin concentration of at least about 1.5 mg/dL.

21. The method of claim 12, wherein the rehydrated plasma product has a pH between about 5.5 and about 7.8.

22. (canceled)

23. (canceled)

24. The method of claim 12, wherein
rehydrating comprises rehydrating over a period of between 1 and 3 minutes.

25. The method of claim 12, wherein
rehydrating comprises rehydrating over a period of less than 2 minutes.

26. (canceled)

27. (canceled)

28. The method of claim 2, wherein the plasma product is stable for at least two years at a temperature of about 15° F. to about 140° F.

29. The method of claim 2, wherein the plasma product is stable for at least two years at a temperature of about −9.5° C. to about 60° C.

30. A plasma product produced by the method of claim 2.

31. A rehydrated plasma product produced by the method of claim 2.

32. A method of treating a subject, the method comprising:
rehydrating a plasma product produced by the method of claim 2 to form a rehydrated plasma product; and
administering the rehydrated plasma product to the subject.

33. (canceled)

34. The method of claim 32, wherein the subject has undergone trauma, has a coagulopathy, or a combination thereof.

35. (canceled)

36. The method of claim 32, wherein the subject is a canine subject or a human subject.

37. (canceled)

38. The method of claim 2, wherein the average particle size of the plasma product is about 0.1 to about 1.0 mm.

39. The method of claim 2, wherein in the method further comprises packaging the plasma product in an ethylene vinyl acetate bag.
